(19) United States
(12) Patent Application Publication
(10) Pub. No.: US 2013/0178756 A1
(43) Pub. Date: Jul. 11, 2013

(54) BREATH DETECTION DEVICE AND BREATH DETECTION METHOD

(71) Applicant: FUJITSU LIMITED, Kawasaki-shi (JP)

(72) Inventors: Masanao SUZUKI, Yokohama (JP); Masakiyo TANAKA, Hachioji (JP); Yasuji OTA, Yokohama (JP)

(73) Assignee: FUJITSU LIMITED, Kawasaki-shi (JP)

(21) Appl. No.: 13/780,274

(22) Filed: Feb. 28, 2013

Related U.S. Application Data
(63) Continuation of application No. PCT/JP2010/066959, filed on Sep. 29, 2010.

(51) Int. Cl. A61B 5/08; A61B 7/00; A61B 5/00

(52) U.S. Cl. CPC A61B 5/0816; A61B 5/7246; A61B 7/003
USPC 600/529

(57) ABSTRACT

Whether a breath sound is contained in a current frame is determined by using a characteristic that a breath sound is small in autocorrelation and large in cross-correlation. Specifically, a harmonic-wave-structure estimating unit finds autocorrelation on the basis of a frequency spectrum of the current frame. A cross-correlation estimating unit finds cross-correlation between the frequency spectrum of the current frame and a frequency spectrum of a previous frame containing a breath sound. A breath detecting unit compares a value of a constant multiple of a value of the autocorrelation with a value of the cross-correlation, and, when the value of the cross-correlation is larger, determines that a breath sound is contained in the current frame.

### CROSS-REFERENCE TO RELATED APPLICATION

This application is a continuation of International Application No. PCT/JP2010/066959, filed on Sep. 29, 2010, the entire contents of which are incorporated herein by reference.

### FIELD

The embodiment discussed herein is directed to a breath detection device and a breath detection method.

### BACKGROUND

In recent years, “sleep apnea”, which is cessation of breathing during sleep, is attracting attention, and it is hoped that a breathing state during sleep is detected accurately and easily. Conventional technologies for breath detection include a technology to perform frequency conversion of input voice of a subject and compare the magnitude of each frequency component with a threshold, thereby detecting sleeper's breathing, snoring, and a roaring sound, etc.

As another conventional technology for breath detection, there is a technology to collect sounds around a subject while the subject is sleeping and determine a period in which there is a sound as a period in which the subject is breathing. In this conventional technology, a cycle of appearance of periods in which there is a sound is detected as the pace of breathing, and, if there is no sound at timing of breathing, this period in which there is no sound is detected as an apnea period. These related-art examples are described, for example, in Japanese Laid-open Patent Publication No. 2007-289660, and Japanese Laid-open Patent Publication No. 2009-219713

However, the above-mentioned conventional technologies have a problem that it is not possible to detect a breath sound accurately.

In the technology to detect subject's breathing by comparing the magnitude of each frequency component with a fixed threshold, due to the influence of a noise around the subject, it may be incorrectly determined that the subject is breathing. Furthermore, in the technology to determine subject's breathing on the basis of whether there is a sound, it is based on the premise that sounds collected from the subject do not include any noises; therefore, it is not possible to detect a breath sound accurately in an environment in which noise occurs.

### SUMMARY

According to an aspect of an embodiment, a breath detection device includes a memory and a processor coupled to the memory. The processor executes a process including: first calculating a frequency spectrum that associates each frequency with signal strength with respect to the frequency, by dividing an input sound signal into multiple frames and performing frequency conversion of each of the frames; shifting a frequency spectrum of a given frame calculated to a frequency direction; second calculating a first similarity indicating how well-matched the before-shifted frequency spectrum and the after-shifted frequency spectrum are; third calculating a second similarity by finding cross-correlation between the frequency spectrum of the given frame and a frequency spectrum of a frame previous to the given frame; and determining whether the frequency spectrum of the given frame indicates breath on the basis of the first similarity and the second similarity.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating a configuration of a breath detection device according to a present embodiment;

FIG. 2 is a diagram for explaining a method to calculate an autocorrelation;

FIG. 3 is a diagram illustrating an example of autocorrelation;

FIG. 4 is a diagram illustrating a frequency spectrum of voice;

FIG. 5 is a diagram illustrating a frequency spectrum of a breath sound;

FIG. 6 is a diagram for explaining cross-correlation of voice;

FIG. 7 is a diagram for explaining cross-correlation of a breath sound;

FIG. 8 is a diagram illustrating respective relations between autocorrelation and cross-correlation of voice and a breath sound;

FIG. 9 is a diagram illustrating an example of a relation between time and cross-correlation;

FIG. 10 is a diagram illustrating an example of a frequency spectrum of voice and a frequency spectrum of breath;

FIG. 11 is a diagram illustrating an example of autocorrelation of voice and autocorrelation of breath;

FIG. 12 is a diagram illustrating an example of cross-correlation of voice and cross-correlation of breath; and

FIG. 13 is a flowchart illustrating a procedure of a process performed by the breath detection device.

### DESCRIPTION OF EMBODIMENTS

Preferred embodiments of the present invention will be explained with reference to accompanying drawings. Incidentally, the present invention is not limited to the embodiment.

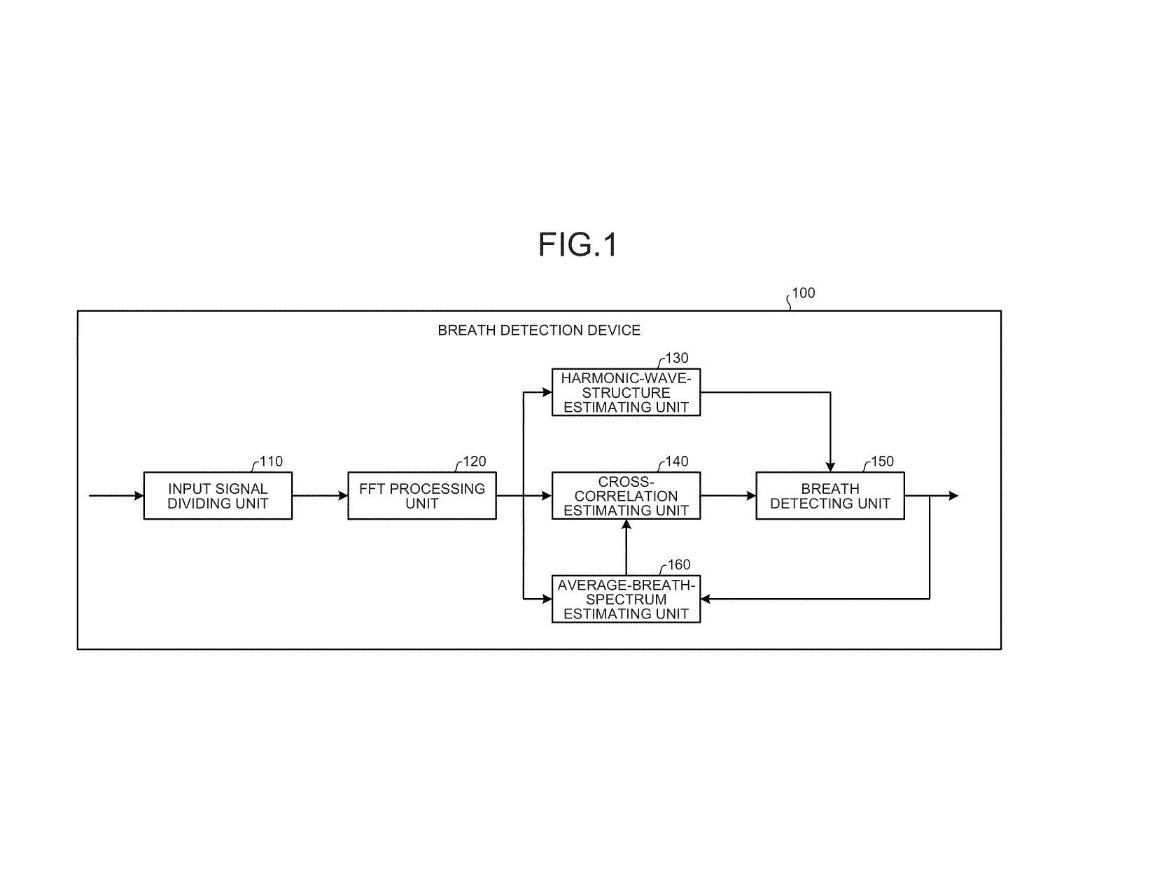

A configuration of the breath detection device according to the present embodiment is explained. FIG. 1 is a diagram illustrating the configuration of the breath detection device according to the present embodiment. As illustrated in FIG. 1, a breath detection device 100 includes a input signal dividing unit 110, a Fast Fourier Transform (FFT) processing unit 120, a harmonic-wave-structure estimating unit 130, a cross-correlation estimating unit 140, a breath detecting unit 150, and an average-breath-spectrum estimating unit 160.

The input signal dividing unit 110 is a processing unit that divides an input signal into multiple frames. The input signal dividing unit 110 outputs the divided frames to the FFT processing unit 120 in chronological order. The input signal is, for example, a sound signal of a sound around a subject collected through a microphone.

The input signal dividing unit 110 divides an input signal into as many frames as the predetermined number N of samples. N is a natural number. The divided nth frame of the input signal is referred to as xn(t). Incidentally, it is provided that t=0, 1, . . . , N−1.

The FFT processing unit 120 is a processing unit that extracts which and how many frequency components an input signal contains, thereby calculating a frequency spectrum. The FFT processing unit 120 outputs the frequency spectrum to the harmonic-wave-structure estimating unit 130, the cross-correlation estimating unit 140, and the average-breath-spectrum estimating unit 160.

Here, a frequency spectrum of an input signal xn(t) is referred to as s(f), provided that f=0, 1, . . . , K−1. K denotes the number of FFT points. When a sampling frequency of input signal is 16 kHz, a value of K is, for example, 256.

When a real part is denoted by Re(f), and an imaginary part is denoted by Im(f), the frequency spectrum s(f) calculated by the FFT processing unit 120 can be expressed by equation (1).

s(f)=|Re(f)²+Im(f)²| (1)

The harmonic-wave-structure estimating unit 130 is a processing unit that finds autocorrelation of a frequency spectrum. The harmonic-wave-structure estimating unit 130 finds autocorrelation Acor(d) on the basis of equation (2).

$$Acor ( d ) = \frac{{\sum }_{f = 0}^{K - 1 - d} s ( f ) \cdot s ( f + d )}{{\sum }_{f = 0}^{K - 1 - d} {s ( f )}^{2}} \qquad ( 2 )$$

In equation (2), d denotes a variable representing a delay. When a sampling frequency of input signal is 16 kHz, and the number of FFT points is 256, a value of a delay d is 6 to 20. The harmonic-wave-structure estimating unit 130 varies a value of d from 6 to 20 sequentially, and finds an autocorrelation Acor(d) with respect to each of the different delays d. The harmonic-wave-structure estimating unit 130 finds the maximum autocorrelation Acor(d1) in the autocorrelations Acor(d). Here, d1 denotes a delay resulting in the maximum autocorrelation. The harmonic-wave-structure estimating unit 130 outputs the autocorrelation Acor(d1) to the breath detecting unit 150.

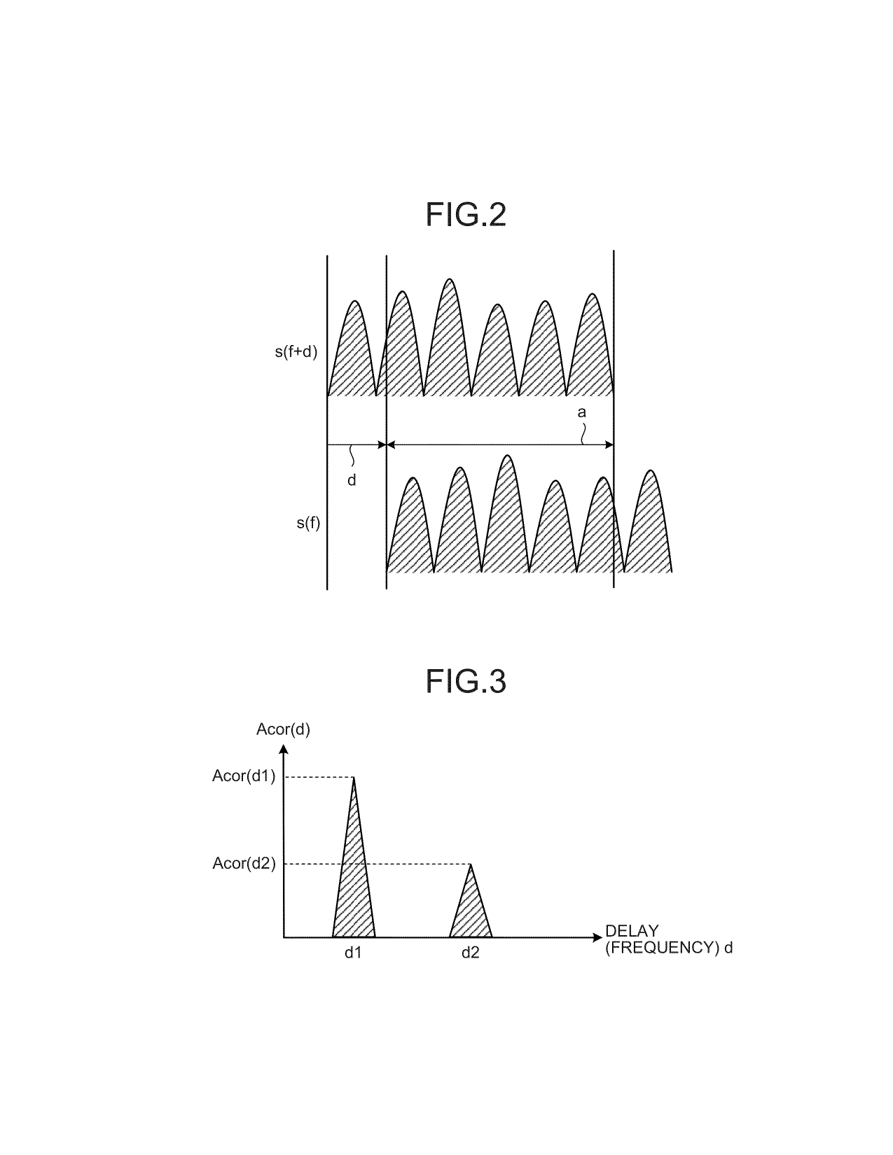

A method to calculate an autocorrelation is explained. FIG. 2 is a diagram for explaining a method to calculate an autocorrelation. As illustrated in FIG. 2, an autocorrelation is obtained by calculating the sum of products of a frequency spectrum s(f+d) and a frequency spectrum s(f) delayed by d from the frequency spectrum s(f+d). A range a in FIG. 2 corresponds to an autocorrelation calculating range.

FIG. 3 is a diagram illustrating an example of autocorrelation. The vertical axis in FIG. 3 indicates a value of autocorrelation, and the horizontal axis corresponds to a delay d. When an autocorrelation Acor(d1) with respect to a delay d1 is compared with an autocorrelation Acor(d2) with respect to a delay d2, the autocorrelation Acor(d1) with respect to the delay d1 is larger. Therefore, the autocorrelation Acor(d1) is a maximum value. As will be described below, a value of autocorrelation differs between when voice is contained in an input signal and when breath is contained in an input signal.

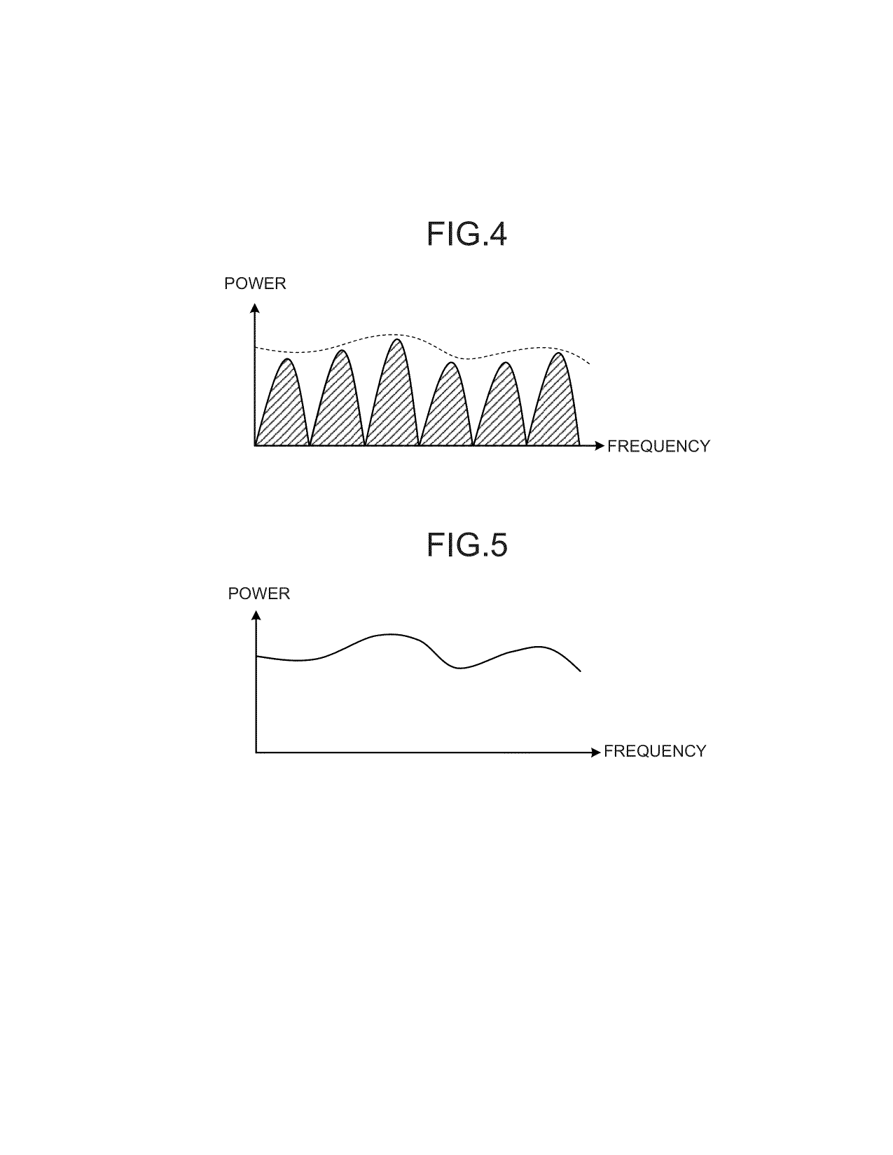

FIG. 4 is a diagram illustrating a frequency spectrum of voice. The vertical axis in FIG. 4 indicates power corresponding to the magnitude of a frequency component, and the horizontal axis indicates frequency. As voice is accompanied by vocal cord vibration, voice has a harmonic wave structure. Therefore, a frequency spectrum shifted to a frequency direction and a before-shifted frequency spectrum are well-matched, and a value of autocorrelation is large.

FIG. 5 is a diagram illustrating a frequency spectrum of a breath sound. The vertical axis in FIG. 5 indicates power corresponding to the magnitude of a frequency component, and the horizontal axis indicates frequency. As breath is not accompanied by vocal cord vibration, breath does not have a harmonic wave structure. Therefore, a frequency spectrum shifted to a frequency direction and a before-shifted frequency spectrum are not well-matched, and a value of autocorrelation is small.

Incidentally, the harmonic-wave-structure estimating unit 130 can find an autocorrelation on the basis of equation (3) instead of equation (2). By using equation (3), the influence of offset of the frequency spectrum s(f) can be eliminated. It is provided that s(−1)=0.

$$Acor ( d ) = \frac{{\sum }_{f = 0}^{K - 1 - d} ( s ( f ) - s ( f - 1 ) ) ( s ( f + d ) - s ( f - 1 + d ) )}{{\sum }_{f = 0}^{K - 1 - d} {( s ( f ) - s ( f - 1 ) )}^{2}} \qquad ( 3 )$$

To return to the explanation of FIG. 1, the cross-correlation estimating unit 140 is a processing unit that finds a cross-correlation between an average frequency spectrum of frequency spectra of previous frames containing a breath sound and a frequency spectrum of a current frame. The cross-correlation estimating unit 140 finds a cross-correlation Ccor(n) on the basis of equation (4). The cross-correlation estimating unit 140 outputs the cross-correlation Ccor(n) to the breath detecting unit 150.

$$Ccor ( n ) = \frac{{\sum }_{f = 0}^{K - 1} {s}_{ave} ( f ) \cdot s ( f )}{{\sum }_{f = 0}^{K - 1} {s ( f )}^{2}} \qquad ( 4 )$$

In equation (4), sₐᵥₑ(f) denotes an average frequency spectrum of frequency spectra of previous frames containing a breath sound. The average frequency spectrum is hereinafter referred to as the average breath spectrum. The cross-correlation estimating unit 140 acquires the average breath spectrum sₐᵥₑ(f) from the average-breath-spectrum estimating unit 160.

When the same frequency spectral feature periodically appears as seen in breath, a value of cross-correlation is large. On the other hand, when the same frequency spectral feature does not periodically appear as seen in voice, a value of cross-correlation is small.

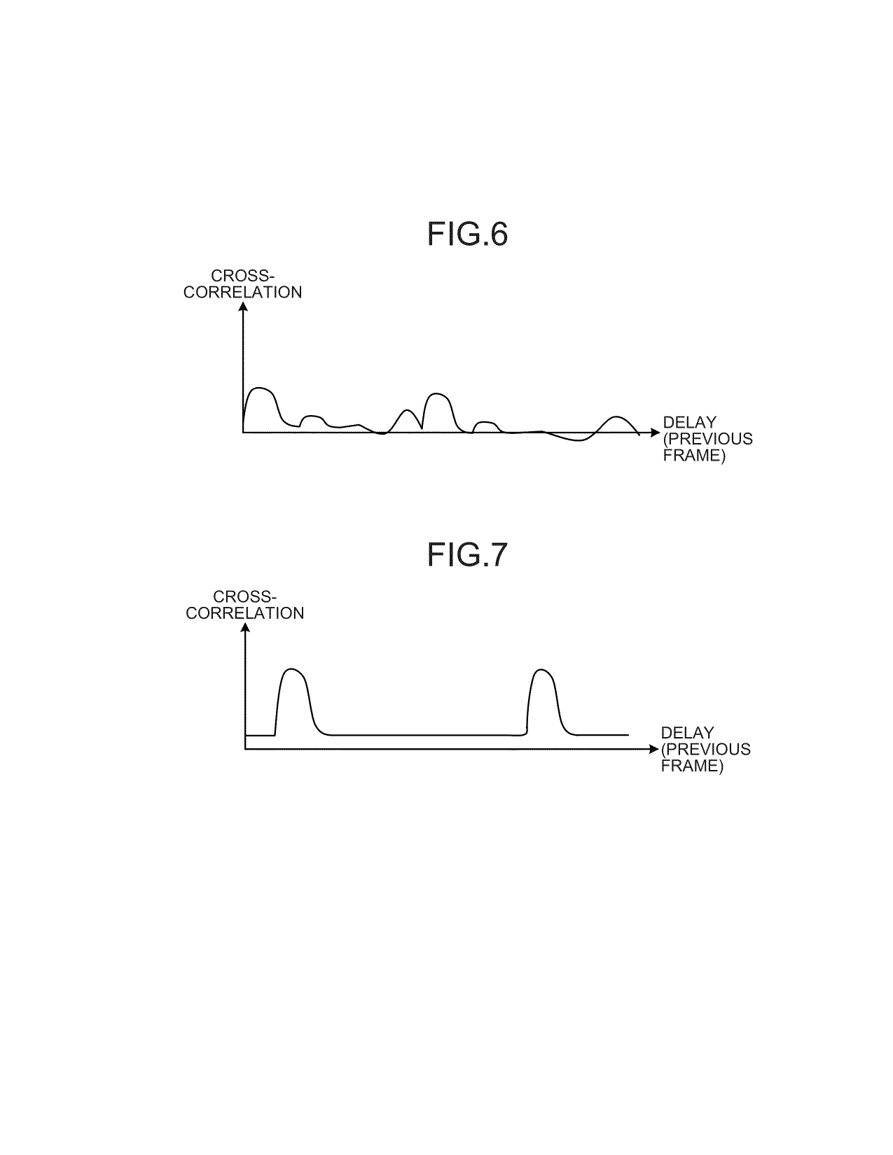

FIG. 6 is a diagram for explaining cross-correlation of voice. The vertical axis in FIG. 6 indicates a value of cross-correlation, and the horizontal axis indicates a delay of a previous frame to be compared with a current frame. As illustrated in FIG. 6, a value of cross-correlation of voice is small.

FIG. 7 is a diagram for explaining cross-correlation of a breath sound. The vertical axis in FIG. 7 indicates a value of cross-correlation, and the horizontal axis indicates a delay of a previous frame to be compared with a current frame. As illustrated in FIG. 7, a value of cross-correlation of a breath sound is large.

Incidentally, the cross-correlation estimating unit 140 can find a cross-correlation on the basis of equation (5) instead of equation (4). By using equation (5), the influence of offset of the frequency spectrum s(f) can be eliminated. It is provided that s(−1)=sₐᵥₑ(−1)=0.

$$Ccor ( n ) = \frac{{\sum }_{f = 0}^{K - 1} ( {s}_{ave} ( f ) - {s}_{ave} ( f - 1 ) ) ( s ( f ) - s ( f - 1 ) )}{{\sum }_{f = 0}^{K - 1} {( s ( f ) - s ( f - 1 ) )}^{2}} \qquad ( 5 )$$

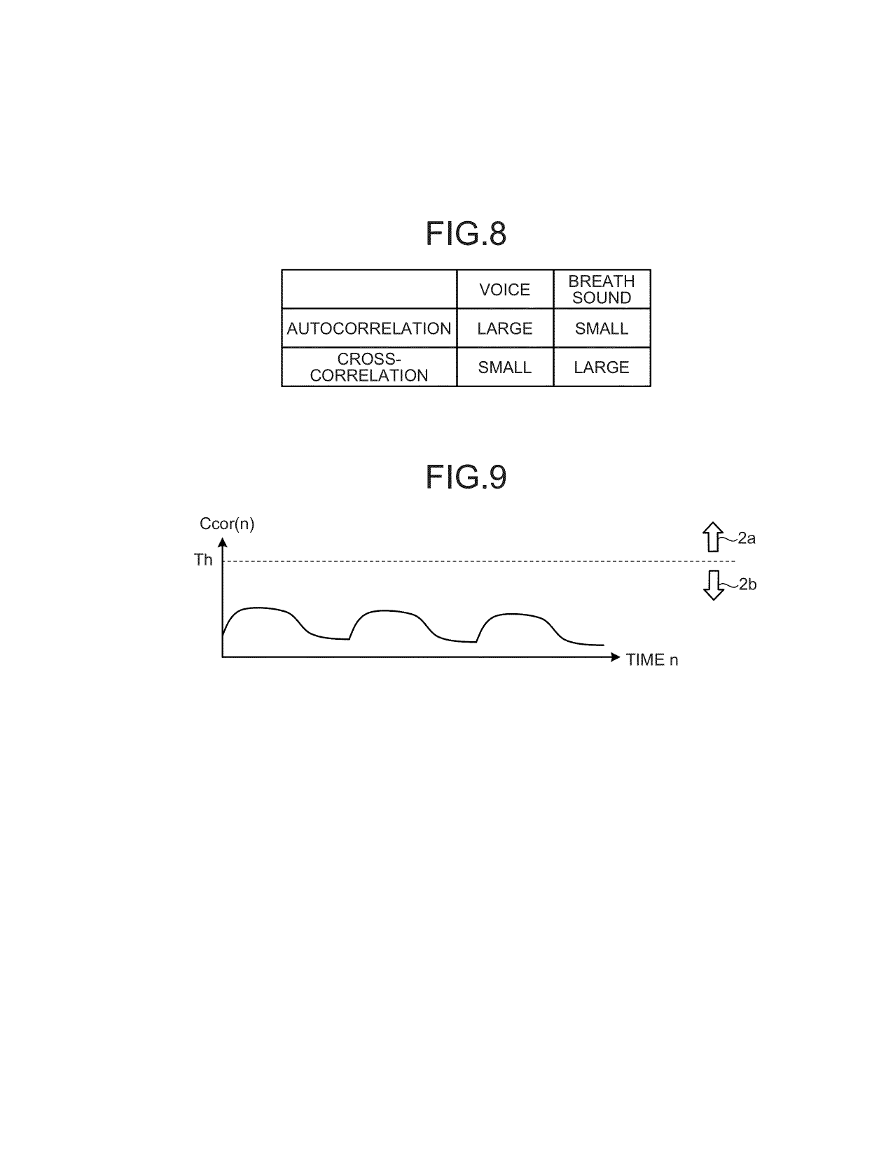

The breath detecting unit 150 is a processing unit that determines whether a breath sound is contained in a current frame on the basis of the autocorrelation Acor(d1) and the cross-correlation Ccor(n). FIG. 8 is a diagram illustrating respective relations between autocorrelation and cross-correlation of voice and a breath sound. As illustrated in FIG. 8, autocorrelation of voice is large, cross-correlation of voice is small. On the other hand, autocorrelation of a breath sound is small, cross-correlation of a breath sound is large. Using the relations illustrated in FIG. 8, the breath detecting unit 150 determines whether a breath sound is contained in a current frame. Namely, when the autocorrelation Acor(d1) and the cross-correlation Ccor(n) are in a relation of cross-correlation Ccor(n)>autocorrelation Acor(d1), the breath detecting unit 150 determines that a breath sound is contained in the current frame. A process performed by the breath detecting unit 150 is explained in detail below.

The breath detecting unit 150 finds a determination threshold Th on the basis of equation (6). In equation (6), β is a constant, and is set to a value ranging from 1 to 10.

Th=β×Acor(d1) (6)

After finding the threshold Th, the breath detecting unit 150 compares a value of Ccor(n) with the threshold Th, and, when a value of Ccor(n) is larger than the threshold Th, determines that a breath sound is contained in the current frame. On the other hand, when a value of Ccor(n) is equal to or smaller than the threshold Th, the breath detecting unit 150 determines that a breath sound is not contained in the current frame.

FIG. 9 is a diagram illustrating an example of a relation between time and cross-correlation. The vertical axis in FIG. 9 indicates cross-correlation Ccor(n), and the horizontal axis in FIG. 9 indicates time. When a value of Ccor(n) is in an area 2a exceeding the threshold Th, the breath detecting unit 150 determines that it is a breath sound; on the other hand, when a value of Ccor(n) is in an area 2b not exceeding the threshold Th, the breath detecting unit 150 determines that it is a sound other than a breath sound.

When the breath detecting unit 150 has determined that a breath sound is contained in the current frame, the breath detecting unit 150 outputs the current frame to the average-breath-spectrum estimating unit 160.

The average-breath-spectrum estimating unit 160 is a processing unit that averages frames containing a breath sound, thereby calculating an average breath spectrum sₐᵥₑ(f). The average-breath-spectrum estimating unit 160 updates the average breath spectrum sₐᵥₑ(f) on the basis of equation (7), and outputs the updated average breath spectrum to the cross-correlation estimating unit 140. In equation (7), α is a constant, and is set to a value ranging from 0 to 1.

sₐᵥₑ(f)=α·sₐᵥₑ(f)+(1−α)·s(f) (7)

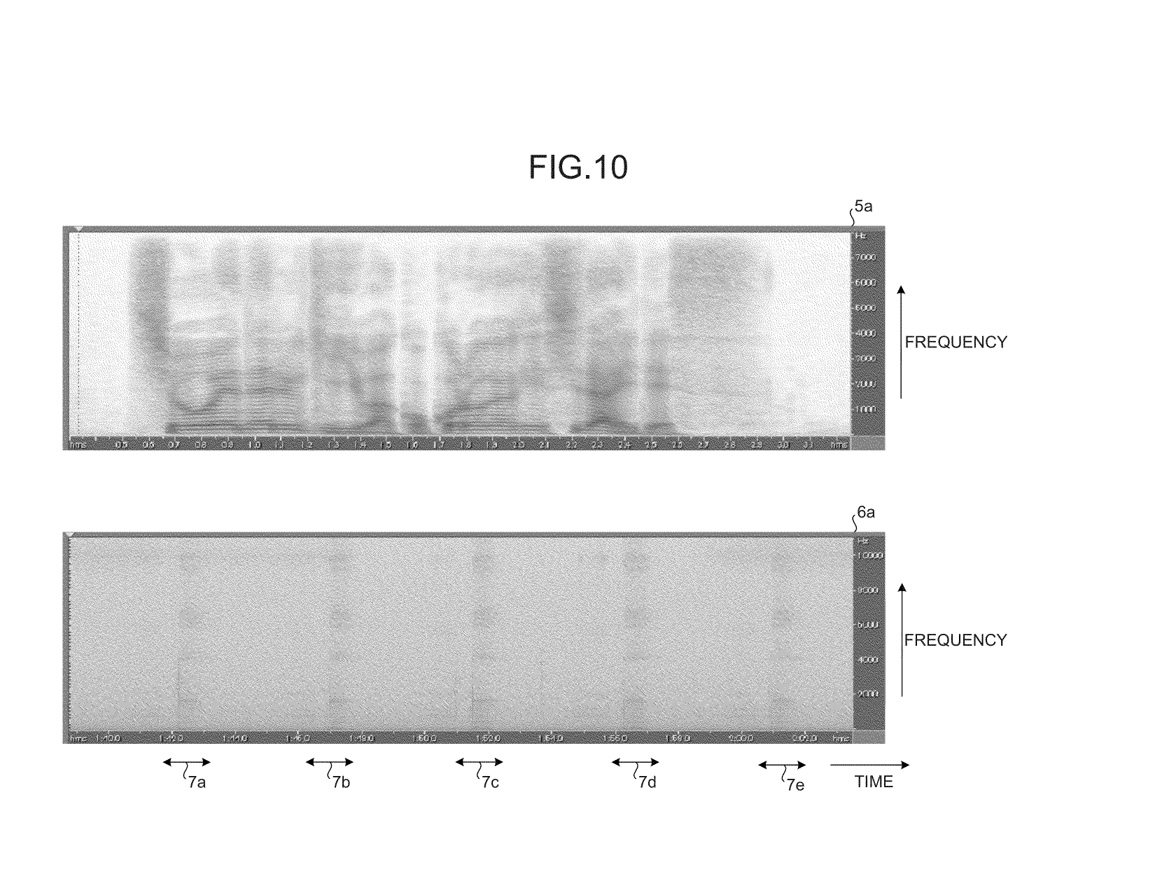

Subsequently, a frequency spectrum of voice and a frequency spectrum of breath are explained by comparison. FIG. 10 is a diagram illustrating an example of a frequency spectrum of voice and a frequency spectrum of breath. An upper diagram in FIG. 10 illustrates a frequency spectrum 5a of voice, and a lower diagram illustrates a frequency spectrum 6a of breath. The horizontal axis of the diagrams is the time axis, and the vertical axis indicates the magnitude of a frequency.

In the frequency spectrum 5a of voice, frequency signals are irregularly generated. On the other hand, in the frequency spectrum 6a of breath, frequency signals are regularly generated. In the example illustrated in FIG. 10, frequency signals are generated in time periods 7a to 7e.

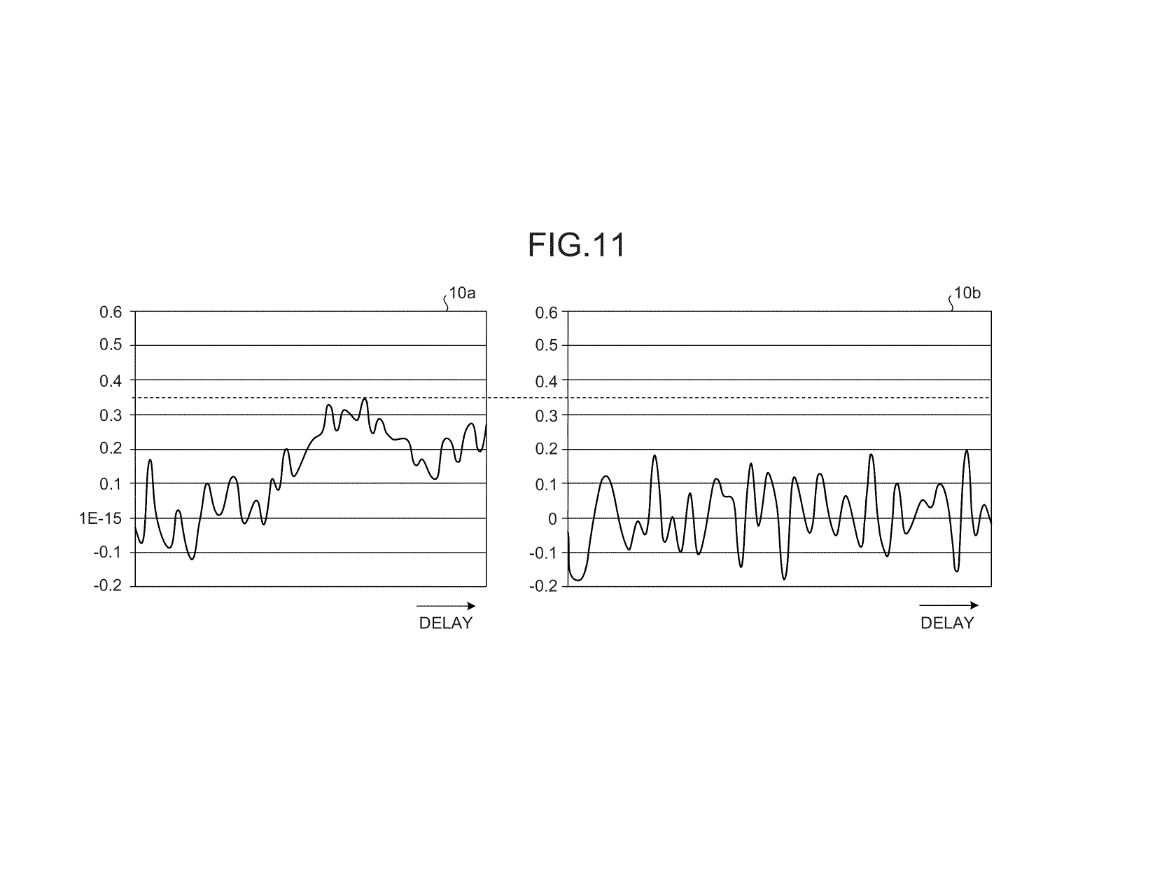

Subsequently, autocorrelation of voice and autocorrelation of breath are explained by comparison. FIG. 11 is a diagram illustrating an example of autocorrelation of voice and autocorrelation of breath. A diagram on the left side of FIG. 11 illustrates autocorrelation 10a of voice, and a diagram on the right side illustrates autocorrelation 10b of breath. The horizontal axis of the diagrams indicates a delay, and the vertical axis indicates the magnitude of an autocorrelation.

In the autocorrelation 10a of voice, the maximum value of autocorrelation is 0.35. On the other hand, in the autocorrelation 10b of breath, the maximum value of autocorrelation is 0.2. Therefore, the maximum value of the autocorrelation 10a of voice is larger than the maximum value of the autocorrelation 10b of breath.

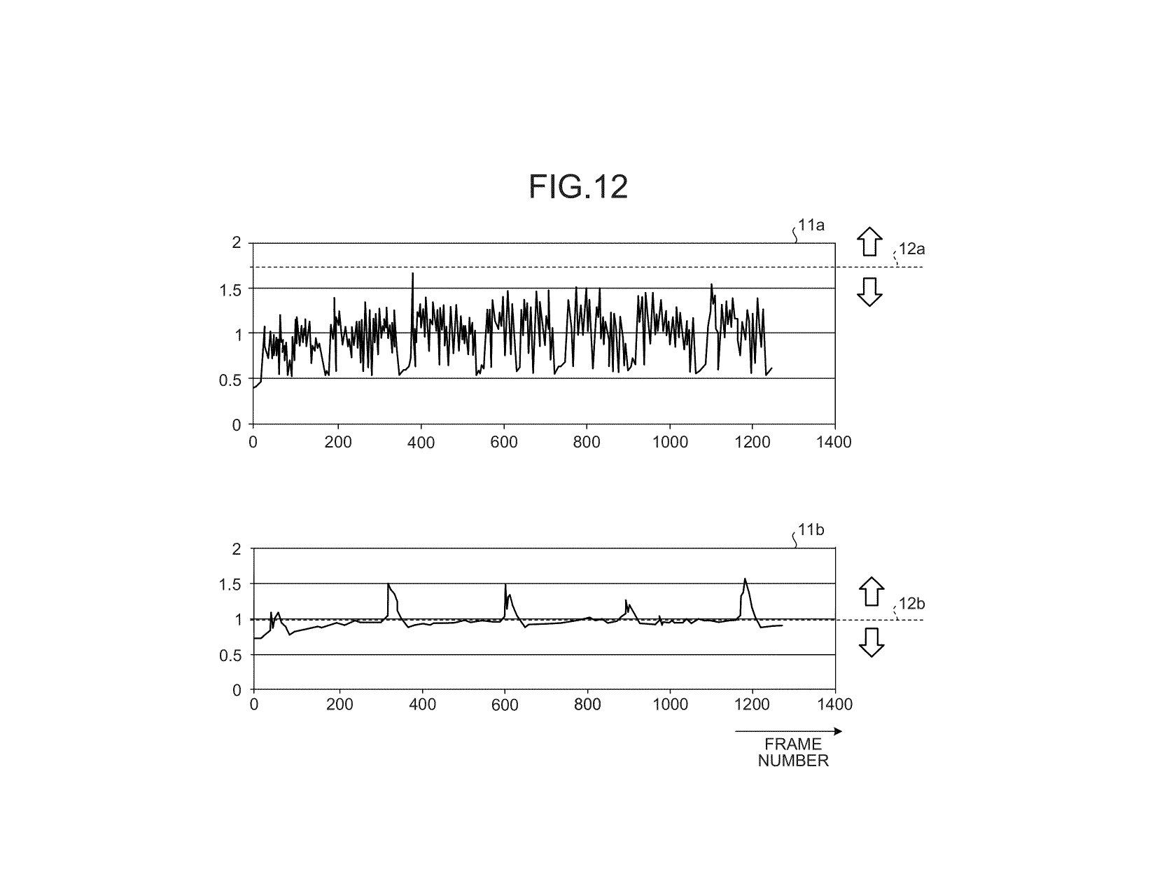

Subsequently, cross-correlation of voice and cross-correlation of breath are explained by comparison. FIG. 12 is a diagram illustrating an example of cross-correlation of voice and cross-correlation of breath. An upper diagram in FIG. 12 illustrates cross-correlation 11a of voice, and a lower diagram illustrates cross-correlation 11b of breath. The horizontal axis of the diagrams indicates a frame number, and the vertical axis indicates the magnitude of a cross-correlation.

A threshold 12a of the cross-correlation 11a of voice is a threshold calculated on the basis of autocorrelation of voice. For example, when the maximum value of autocorrelation of voice is 0.35 and a value of p is 5.0, the threshold 12a is 1.75. As illustrated in FIG. 12, the cross-correlation 11a of voice does not exceed the threshold 12a.

A threshold 12b of the cross-correlation 11b of breath is a threshold calculated on the basis of autocorrelation of breath. For example, when the maximum value of autocorrelation of breath is 0.20 and a value of p is 5.0, the threshold 12b is 1.00. As illustrated in FIG. 12, the cross-correlation 11b of breath exceeds the threshold 12b at timing of breath.

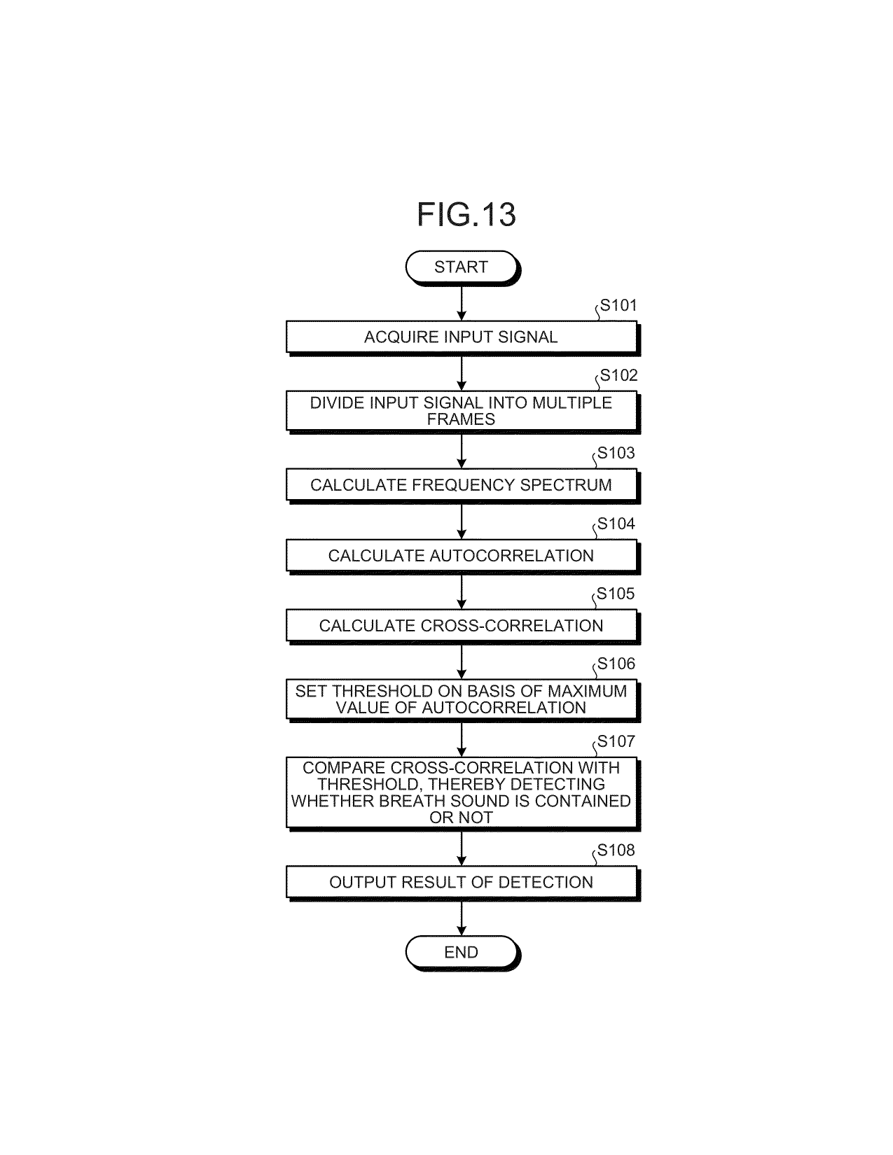

Subsequently, a procedure of a process performed by the breath detection device 100 is explained. FIG. 13 is a flowchart illustrating the procedure of the process performed by the breath detection device. The process illustrated in FIG. 13 is performed, for example, when an input signal is input to the breath detection device 100.

As illustrated in FIG. 13, the breath detection device 100 acquires an input signal (Step S101), and divides the input signal into multiple frames (Step S102). The breath detection device 100 calculates a frequency spectrum (Step S103), and calculates autocorrelation (Step S104).

The breath detection device 100 calculates cross-correlation (Step S105), and determines a threshold on the basis of the maximum value of the autocorrelation (Step S106). The breath detection device 100 compares the cross-correlation with the threshold, thereby detecting whether a breath sound is contained in the input signal (Step S107), and outputs a result of the detection (Step S108).

Subsequently, the effects of the breath detection device 100 according to the present embodiment are explained. When a breath sound is contained in an input signal, autocorrelation is small and cross-correlation is large. This characteristic is applied equally in a case where a noise is contained in the input signal. Therefore, without being affected by noise, the breath detection device 100 can accurately detect a frame containing a breath sound by determining whether a breath sound is contained in a frame on the basis of autocorrelation and cross-correlation of an input signal.

The breath detection device 100 according to the present embodiment finds an average breath spectrum by weighted-averaging frequency spectra of frames containing a breath sound, and finds cross-correlation between a frequency spectrum of a current frame and the average breath spectrum. Therefore, it is possible to eliminate error between frequency spectra of previous frames containing a breath sound and find cross-correlation accurately.

The breath detection device 100 according to the present embodiment compares a value of β times a value of autocorrelation with a value of cross-correlation, thereby determining whether a breath sound is contained in a current frame. By adjusting a value of β, whether a breath sound is contained in a current frame can be accurately determined in various environments.

Incidentally, components of the breath detection device 100 illustrated in FIG. 1 are functionally conceptual ones, and do not always have to be physically configured as illustrated in FIG. 1. Namely, the specific forms of division and integration of components of the breath detection device 100 are not limited to that is illustrated in FIG. 1, and all or some of the components can be configured to be functionally or physically divided or integrated in arbitrary units depending on respective loads and use conditions, etc. For example, the harmonic-wave-structure estimating unit 130, the cross-correlation estimating unit 140, the breath detecting unit 150, and the average-breath-spectrum estimating unit 160 can be mounted in different devices, respectively, and the devices can determine whether a breath sound is contained in a frame in cooperation with one another.

A breath detection device discussed herein can detect a breath sound accurately.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention has been described in detail, it should be understood that the various changes, substitutions, and alterations could be made hereto without departing from the spirit and scope of the invention.

What is claimed is:

1. A breath detection device including:
a memory; and
a processor coupled to the memory, wherein the processor executes a process comprising:
first calculating a frequency spectrum that associates each frequency with signal strength with respect to the frequency, by dividing an input sound signal into multiple frames and performing frequency conversion of each of the frames;
shifting a frequency spectrum of a given frame calculated in a frequency direction;
second calculating a first similarity indicating how well-matched the before-shifted frequency spectrum and the after-shifted frequency spectrum are;
third calculating a second similarity by finding cross-correlation between the frequency spectrum of the given frame and a frequency spectrum of a frame previous to the given frame; and
determining whether the frequency spectrum of the given frame indicates breath on the basis of the first similarity and the second similarity.

2. The breath detection device according to claim 1, wherein
the second calculating includes finding autocorrelation of the frequency spectrum of the given frame.

3. The breath detection device according to claim 1, wherein
the third calculating includes finding cross-correlation between a frequency spectrum obtained by weighted-averaging frequency spectra of frames containing a breath sound out of frames previous to the given frame and the frequency spectrum of the given frame.

4. The breath detection device according to claim 3, wherein
the determining includes determining that the frequency spectrum of the given frame indicates breath, when a value of the second similarity is larger than a value of a constant multiple of the first similarity.

5. A breath detection method executed by a breath detection device, the breath detection method comprising:
first calculating, using a processor, a frequency spectrum that associates each frequency with signal strength with respect in the frequency, by dividing an input sound signal into multiple frames and performing frequency conversion of each of the frames;
shifting, using the processor, a frequency spectrum of a given frame calculated to a frequency direction;
second calculating, using the processor, a first similarity indicating how well-matched the before-shifted frequency spectrum and the after-shifted frequency spectrum are;
third calculating, using the processor, a second similarity by finding cross-correlation between the frequency spectrum of the given frame and a frequency spectrum of a frame previous to the given frame; and
determining, using the processor, whether the frequency spectrum of the given frame indicates breath on the basis of the first similarity and the second similarity.

6. The breath detection method according to claim 5, wherein
the second calculating includes finding autocorrelation of the frequency spectrum of the given frame.

7. The breath detection method according to claim 5, wherein
the third calculating includes finding cross-correlation between a frequency spectrum obtained by weighted-averaging frequency spectra of frames containing a breath sound out of frames previous to the given frame and the frequency spectrum of the given frame.

8. The breath detection method according to claim 7, wherein
the determining includes determining that the frequency spectrum of the given frame indicates breath, when a value of the second similarity is larger than a value of a constant multiple of the first similarity.
